(19) United States
(12) Patent Application Publication
(10) Pub. No.: US 2005/0246006 A1
(43) Pub. Date: Nov. 3, 2005

(54) Electrical nerve stimulation device

(75) Inventor: Susan Romao-Duarte Daniels, Haywards Heath (GB)

Correspondence Address: ROTHWELL, FIGG, ERNST & MANBECK, P.C., 1425 K STREET, N.W., SUITE 800, WASHINGTON, DC, 20005, US

(73) Assignee: Algotec Limited, Crawley (GB)

(21) Appl. No.: 10/954,653

(22) Filed: Oct. 1, 2004

(30) Foreign Application Priority Data
Apr. 30, 2004 (GB) GB 0409769.7
Sep. 7, 2004 (GB) GB 0419837.0

(51) Int. Cl. A61N 1/18

(52) U.S. Cl. 607/117; 607/46; 607/36

(57) ABSTRACT

The present invention relates to an electrical nerve stimulation device, along with associated methods of use and manufacture. A particular, but not exclusive, application of the invention is subcutaneous electrical nerve stimulation (SENS) for relief of neuropathic pain.

### FIELD OF THE INVENTION

This invention relates to an electrical nerve stimulation device, along with associated methods of use and manufacture. A particular, but not exclusive, application of the invention is subcutaneous electrical nerve stimulation (SENS) for relief of neuropathic pain.

### BACKGROUND OF THE INVENTION

Some pain is nociceptive, e.g. caused when nociceptors or the ends of nerve fibres located in tissues of a human or animal body are stimulated to cause nerve fibres to transmit pain messages. One type of nociceptive pain is known as hyperalgesia (or fast pain) and involves the transmission of pain messages along nerve fibres known as C fibres to portions of the spinal cord or main peripheral nerves known as Rexed Laminae 1 and 2, which pass the pain messages to the sensory cortex. It is known that nerve fibres can be stimulated using electrical pulses to inhibit passage of these pain messages and reduce the sensation of the pain in the affected body area. One method of doing this is called transcutaneous electrical nerve stimulation (TENS).

TENS involves the application of electrical pulses to the body via electrode pads disposed on the surface of the skin. The electrical pulses pass through the skin and stimulate nerves and nerve endings in body tissues under the skin in the region of the electrodes. However, whilst this has proved to be effective in alleviating pain such as back pain and pain associated with pregnancy and child birth, some patients have increased pain in the presence of TENS therapy. In these patients, C fibre termination at Rexed Laminae 1 and 2 ceases as a result of peripheral nerve damage and is replaced at Rexed Laminae 1 and 2 by Ab fibres. The Ab fibres projecting into Rexed Laminae 1 and 2 cause an exaggerated nociceptive response to what are normally innocuous stimuli.

Another electrical nerve stimulation treatment is known as spinal cord stimulation (SCS) or dorsal column stimulation (DCS). This involves the application of electrical pulses directly to the spinal cord and can be used to treat both nociceptive pain and neuropathic pain, e.g. pain resulting from disease or dysfunction of peripheral nerves. Electrodes may be surgically implanted close to the spinal cord, e.g. in the epidural space and even touching dura mater surrounding the spinal cord. Using these electrodes, electrical pulses are applied to the spinal cord via the epidural space and/or cerebrospinal fluid. This is very effective in providing pain relief. However, implanting the electrodes, e.g. by accessing the epidural space, requires significant invasive surgery. This carries with it the risk of infection and damage to the spinal cord. Other problems with SCS are that it tends to cause paraesthesia (abnormal sensations such as pins and needles) and only relatively large regions of the body can be targeted. In other words, pain in localised regions of the body, and in particular localised regions of the trunk, cannot be effectively targeted using SCS.

In order to try to target more localised regions of the body, another electrical nerve stimulation treatment, known as peripheral nerve stimulation (PNS), has been developed. PNS involves the application of electrical pulses directly to major nerves extending away from the spinal cord, such as the sciatic nerve of the leg. This can provide pain relief more localised than that of SCS. However, PNS still requires significant invasive surgery for the electrodes to be put in place. Indeed, as the precise location of the major nerves extending away from the spinal cord varies from patient to patient, the surgeon may well need to cut away a significant amount of tissue to locate the desired nerve during electrode implantation. This can cause significant trauma to the patient, carries the risk of nerve damage and is generally undesirable.

Electrodes for both SCS and PNS are usually implanted whilst the patient is either under general anesthesia or heavily sedated. The implantation therefore tends to be an inpatient procedure and is expensive in terms of operating room time and bed occupancy. It also takes up resources such as fluoroscopy equipment, which have multiple other uses.

So, more recently, a technique known as subcutaneous electrical nerve stimulation (SENS) has been suggested. SENS involves positioning electrodes just below the skin and can be used to target nerves and nerve endings in very specific regions, including localised regions of the trunk or abdomen. It is thought that SENS causes hyperpolarisation of Ab fibres in the presence of neuropathic pain which can block the transmission of pain.

SENS is less invasive than both SCS and PNS. It has also been found that SENS does not cause the paresthesia of SCS, but rather creates an absence of pain. At the same time, SENS avoids the problem of having to pass electric current through the skin associated with TEN and does not risk the exaggerated nociceptive response associated with TENS. However, SENS is a relatively new treatment and conventional electrical nerve stimulation devices are generally not suitable for use in this type of treatment. Fully effective treatment methods are also yet to be developed. The present invention seeks to overcome these problems.

### SUMMARY OF THE INVENTION

According to a first aspect of the present invention, there is provided an electrical nerve stimulation device, the device comprising:
an electrode lead having electrodes disposed along its length;
an electrical pulse generator connected to the electrode lead for applying electrical pulses to the electrodes; and
a fluid impermeable housing enclosing the connection between the lead and the pulse generator so that the device is a single unit.

According to a second aspect of the present invention, there is provided a method of manufacturing an electrical nerve stimulation device, the method comprising:
connecting an electrode lead having electrodes disposed along its length to an electrical pulse generator for applying pulses of electrical potential to the electrodes; and
enclosing the connection between the lead and the pulse generator in a fluid impermeable housing so that the device is a single unit.

According to a third aspect of the present invention, there is provided an electrical nerve stimulation device that is implantable in a human or animal body, the device comprising:
an electrode lead having electrodes disposed along its length; and
an electrical pulse generator connected to the electrode lead for applying electrical pulses to the electrodes,
wherein the distance along the lead between the pulse generator and the electrode closest to the pulse generator is less than around 5 cm.

According to a fourth aspect of the present invention, there is provided an electrical nerve stimulation device, the device comprising:
an electrode lead having electrodes disposed along its length;
an electrical pulse generator connected to the electrode lead for applying electrical pulses to the electrodes; and
a housing containing the pulse generator, which housing is substantially flat.

According to a fifth aspect of the present invention, there is provided a electrical nerve stimulation device, the device comprising:
an electrode lead having electrodes disposed along its length; and
an electrical pulse generator connected to the electrode lead for applying electrical pulses to the electrodes,
wherein the pulse generator is substantially flexible.

According to a sixth aspect of the present invention, there is provided an electrical nerve stimulation device, the device comprising:
an electrode lead having electrodes disposed along its length; and
an electrical pulse generator connectable to the electrode lead for applying electrical pulses to the electrodes,
wherein the electrode lead has an electronically readable memory for storing data.

According to a seventh aspect of the present invention, there is provided an electrode lead for electrical nerve stimulation, the lead having electrodes disposed along its length and an electronically readable memory for storing data.

According to an eighth aspect of the present invention, there is there is provided an electrical nerve stimulation device, the device comprising:
an electrode lead having electrodes disposed along its length; and
an electrical pulse generator connectable to the electrode lead for applying electrical pulses to the electrodes,
wherein the electrodes each comprise a group of electrical contacts.

According to a ninth aspect of the present invention, there is provided an electrode lead for electrical nerve stimulation, the lead having electrodes that each comprise a group of electrical contacts.

According to a tenth aspect of the present invention, there is therefore provided a method of implanting an electrical nerve stimulation device in a human or animal body, the method comprising:
inserting a needle carrying a sheath into a subcutaneous region;
withdrawing the needle to leave the sheath in place in the subcutaneous region;
inserting an electrode lead of the electrical nerve stimulation device into the sheath; and
removing the sheath from the subcutaneous region to expose the electrode lead in the subcutaneous region.

According to an eleventh aspect of the present invention, there is provided a method of treating pain, the method comprising:
implanting an electrical nerve stimulation device in subcutaneous tissue of a human or animal body; and
applying electrical pulses to the tissue in which the device is implanted via an electrode lead of the device.

Preferred embodiments of the invention will now be described, by way of example only, with reference to the accompanying drawings.

### BRIEF DESCRIPTION OF THE DRAWINGS

FIG. 1 is an illustration of a first embodiment of an electrode lead for electrical nerve stimulation.

FIG. 2 is an illustration of a second embodiment of an electrode lead for electrical nerve stimulation.

FIG. 3 is an illustration of a third embodiment of an electrode lead for electrical nerve stimulation.

FIG. 4A is an illustration of an electrical pulse generator for use with the electrode leads of FIGS. 1 to 3.

FIG. 4B is a sectional view along the line A-A of the electrical pulse generator illustrated in FIG. 4A.

FIG. 5A is an illustration of a first embodiment of an electrical nerve stimulation device of the invention.

FIG. 5B is a sectional view along the line B-B of the electrical nerve stimulation device illustrated in FIG. 5A.

FIG. 6A is an illustration of a second embodiment of an electrical nerve stimulation device of the invention.

FIG. 6B is a sectional view along the line C-C of the electrical nerve stimulation device illustrated in FIG. 6A.

FIG. 7 is an illustration of a control unit for use with the electrical pulse generator of FIGS. 4A and 4B and the electrical nerve stimulation devices of FIGS. 5A to 6B.

FIG. 8 is an illustration of a programming unit for use with the electrical pulse generator of FIGS. 4A and 4B and the electrical nerve stimulation devices of FIGS. 5A to 6B.

FIG. 9A is an illustration of a first embodiment of an introducing instrument for use during insertion of the electrode leads and electrical nerve stimulation devices of the invention in a body.

FIG. 9B is an illustration of the introducing instrument of FIG. 9A with a peel sheath in place.

FIG. 10A is an illustration of a second embodiment of an introducing instrument for use during insertion of the electrode leads and electrical nerve stimulation devices of the invention in a body.

FIG. 10B is an illustration of the introducing instrument of FIG. 10A with a peel sheath in place.

FIG. 11 is an illustration of a marker for use during insertion of the electrode leads and electrical nerve stimulation devices of the invention in a body.

FIG. 12 is an illustration of an area of a body to be treated by insertion of the electrode leads and electrical nerve stimulation devices of the invention.

FIG. 13 is an illustration of the treatment area of FIG. 12 illustrating the positioning of one of the electrical nerve stimulation devices of the invention.

FIG. 14A is an illustration of a first electrical pulse waveform that can be applied by the invention.

FIG. 14B is an illustration of a second electrical pulse waveform that can be applied by the invention.

FIG. 15 is an illustration of a modem for remote programming of the electrical nerve stimulation devices of the invention.

### DETAILED DESCRIPTION OF THE PREFERRED EMBODIMENTS

In one embodiment, both the lead and the pulse generator can be integral to the device, which allows the device to be supplied as a single sealed unit. In other words, the device may be a sealed unit. This has significant advantages over existing electrical nerve stimulation devices, which typically comprise leads and electrical pulse generators supplied separately and connected to one another in situ. For example, the device of the invention can be implanted in a patient more safely and retained in the body for extended periods of time. More specifically, the ingress of bodily fluids or foreign matter into the connection between the lead and the pulse generator is prevented, which significantly reduces the possibility of the device corroding. Furthermore, current leakage at the connection between the lead and the pulse generator is prevented. This avoids the unintended application of electrical pulses to tissues surrounding the connection, which can cause an unpleasant tingling or burning sensation when conventional devices are used. It also reduces the risk of the lead becoming separated from the pulse generator, which can occur in conventional devices.

The pulse generator may have its own housing and the fluid impermeable housing can extend over the connection and at least a portion of the pulse generator's housing. However, it is particularly preferred that the fluid impermeable housing encloses both the pulse generator and the connection. Similarly, whilst the entire electrical nerve stimulation device other than the electrodes can be enclosed by the fluid impermeable housing, it is preferred that only a (small) portion of the lead proximal to the pulse generator is enclosed by the fluid impermeable housing.

The fluid impermeable housing can comprise a variety of materials and designs. The material might be a plastics material for example, and is preferably biocompatible. It is particularly preferred that the housing is silicone. This is convenient as it can be applied to the lead and pulse generator in a molten state to provide a very effective seal. This might be achieved by moulding the housing around the lead and pulse generator for example. Silicone can also be fairly flexible and soft, which improves patient comfort.

It is intended that the device is implantable in a human or animal body. More specifically, the entire device may be implantable. Conventional pulse generators used for SCS and PNS are fairly large, as they require relatively high capacity and therefore large batteries to meet the power requirements of SCS and PNS therapies. It has only therefore previously been possible to implant pulse generators for SCS and PNS in a restricted range of sites, e.g. in the abdomen or upper quadrant of the buttock, as only a few sites can comfortably accommodate a large pulse generator. The site of the pulse generator may therefore be significantly spaced apart from the treatment site. This has meant that the distance between the pulse generator and the electrodes on the lead has been relatively long and that patients have suffered significant trauma in having the lead tunnelled under the skin from the treatment site, e.g. in the epidural space, to meet the pulse generator at the site at which it is implanted, e.g. in the abdominal cavity. However, the applicants have recognised that SENS can be effective with far less power than SCS and PNS, so lower capacity batteries and hence smaller pulse generators can provide effective and long term treatment. This opens up the possibility of positioning the electrical pulse generator close to the treatment site, e.g. in a region close to the skin surface and makes it possible to implant the device in new areas, such as the foot, arm, head or neck etc. So, it is preferred that the distance along the lead between the pulse generator and the electrode closest to the pulse generator is short, e.g. less than around 5 cm.

In other words, the electrical pulse generator may be close to the electrodes on the electrode lead. When the device is implanted, this means that the electrical pulse generator may be close to the treatment site. Such a device can be implanted with significantly less trauma than previous implantable electrical nerve stimulation devices. The lead also tends to have less electrical impedance, with the result that less power is required to deliver a given electrical pulse to the patient, which ultimately extends the battery life of the pulse generator.

Furthermore, when existing devices having a long length of lead between the pulse generator and electrodes are used, it may sometimes be necessary to coil excess lead inside the body. Over time, this can cause to lead fracture inside the body. However, the short distance between the pulse generator and the electrodes of the invention eliminates the need for this excess lead to be implanted in the body and thus reduces the risk of lead fracture.

Another advantage is that the overall size of the device can be smaller than previous devices. However, size reduction has by far the greatest benefit at the pulse generator. In particular, it is preferred that the housing containing the pulse generator is substantially flat.

A flat pulse generator can be implanted close to the skin without significant discomfort to the patient. In one example, the pulse generator may be less than around 7 mm thick. This is sufficiently thin to alleviate discomfort but still allow the pulse generator to house a power supply of sufficient capacity for the device to remain operational for a long period, e.g. around two to seven years for a typical treatment regime. In another example, the pulse generator may be less than around 5 mm thick. This reduced thickness further improves patient comfort and still allows a power supply of sufficient capacity for the device to provide an effective treatment period.

The applicants have also recognised that, particularly when the pulse generator is positioned just under the skin, e.g. at a site such as the foot or neck, it is preferable that that pulse generator is substantially flexible to improve patient comfort. This allows the pulse generator to conform, at least to some extent, to the tissues in which it is implanted.

In one example, this is achieved by the pulse generator comprising a flexible circuit board to which components of the generator are mounted. Similarly the housing of the pulse generator may be flexible, e.g. by being made from a flexible material such as silicone.

Whilst the electrical nerve stimulation device may be a single unit, as mentioned above, the lead and pulse generator are generally manufactured as separate devices initially. Indeed, in other examples of the invention, the lead and pulse generator can be supplied separately rather than as a single unit, e.g. for the purpose of trial stimulation prior to fully implanting a permanent device. The applicants have therefore recognised that it is useful for the pulse generator to be able to identify the type of electrode lead to which it is connected. So, in another example, it is preferred that the electrode lead has an electronically readable memory for storing data.

The data typically includes information about the lead. This might be a serial number, model number or code or specific information about the number of electrodes or such like. This information can be stored in a read only memory for access by the pulse generator. So, the electronically readable memory may comprise a read-only memory (ROM).

In other embodiments, it may be useful to update the data stored in the memory. For example, information about a patient's treatment history may be stored in the memory. In this case, the memory may comprise a writable memory, such as a non-volatile random access memory (RAM).

The electrodes may take a variety of forms. However, it is preferable that the surface area of the contact between the electrodes and the tissues in which they are implanted is a large portion of the surface area of the lead. At the same time, it is preferred that the lead is flexible to aid insertion and allow it to be comfortably accommodated by the patient, e.g. just under the patient's skin. As the electrodes are generally metallic and hence stiff, these desired features can be incompatible. It is therefore preferred that the electrodes each comprise a group of separate electrical contacts.

The contacts of the group may be separate from one another, but connected to each other to form the electrode. For example, the contacts may be connected to each other by a wire inside the electrode lead.

Each contact may be relatively short in comparison to the length of the electrode and the lead between each contact can remain flexible. Thus, each electrode is flexible in comparison to a continuous electrode of the same length.

Each electrode typically has between 2 and 10 contacts. The electrical contacts may extend along the length of the lead substantially between around 2 mm and 5 mm. In other words, they may be around 2 mm to 5 mm in width. They may be spaced apart from one another along the length of the lead by around 3 mm.

The electrical nerve stimulation device or electrode lead may be implanted in the body in a variety of ways. However, as the lead is generally flexible, it is desirable for the implantation of the lead to be assisted by a stiff needle or such like. At the same time, it is desirable to minimise trauma to the patient.

This is effective as the needle need not be any thicker than the lead and need only be inserted over a length similar to the length of the lead. So, trauma to the patient can be minimised. Typically, as the sheath is withdrawn it is torn to remove it from the lead. The sheath is therefore usually thin or tearable.

The invention can be used to relieve various types of pain, depending on the treatment site at which the lead is inserted and the nature of the electrical pulses applied by the lead. However, importantly, the invention allows both the electrode lead and the pulse generator of the electrical nerve stimulation device to be implanted in a subcutaneous region close to the treatment site to treat pain at the site.

The subcutaneous tissue is usually fatty tissue found between the skin and the fascia and muscle tissue underlying the skin. Both an electrical pulse generator and the electrode lead of the device may be implanted in this tissue. Typically, this tissue is around 5 mm or so below the surface of the skin and the device in therefore implanted around 5 mm below the surface of the skin, although this might vary between roughly 2 mm and 20 mm in some cases. Furthermore, the electrode lead of the device may extend into other tissues in some cases.

Generally, the lead is positioned under an area of skin at which the patient experiences greatest allodynia or hyperalgesia. Usually, the lead extends along the major axis of this area. The method therefore typically includes identifying the area of greatest allodynia or hyperalgesia and implanting the lead across the identified area (e.g. along its major axis). This is useful to treat, inter alia, post mastectomy pain, lymphedema, neuropathic chest wall pain, chronic post surgical pain, complex regional pain syndrome (CRPS), neuropathic head, neck and facial pain, neuropathic foot pain, neuropathic abdominal wall pain, neuropathic failed back surgery syndrome (FBSS), angina, migraine, post traumatic cervical neuropathic pain and coccydynia. In some cases, the invention can be used at the same time as SCS. In other examples, the lead can be implanted at specific sites, such as in the inguinal canal to treat post inguinal hernia repair pain, penile/scrotal/testicular pain or vulvadynia.

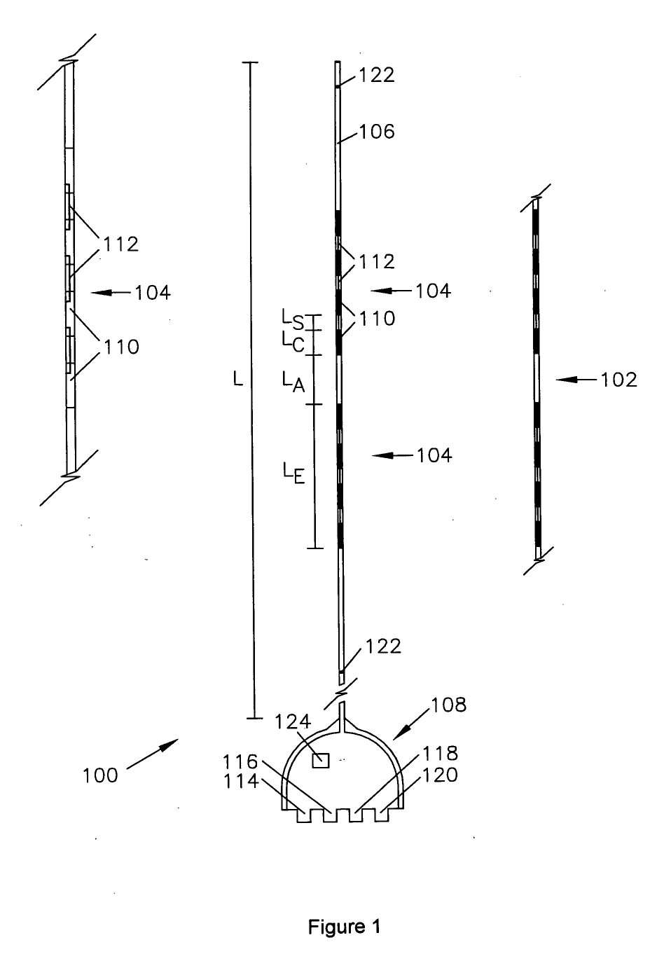

Referring to FIGS. 1 to 3, three embodiments of a temporary electrode lead 100, 200, 300 via which electrical pulses can be applied to a human or animal body are illustrated. Each electrode lead 100, 200, 300 comprises an electrode array 102, 202, 302 of two or more electrodes 104, 204, 304 mounted on an elongate element 106, 206, 306 and a connector 108, 208, 308 positioned at one end of the elongate element 106, 206, 306. The leads 100, 200, 300 are made from a flexible, biocompatible, insulating material, such as polyurethane or polyethylene.

The electrodes 104, 204, 304 each comprise a series of contacts 110, 210, 310 joined to one another by wires 112, 212, 312 inside the elongate elements 106, 206, 306. So, whilst each electrode 104, 204, 304 has multiple contacts 110, 210, 310, it is effectively only a single “electrode” or “contact set”. The contacts 110, 210, 310 are made from a biocompatible conductor, such as a platinum/iridium alloy and are relatively solid and inflexible, in that they extend around the respective leads 100, 200, 300, e.g. they are substantially annular. However, the wires 112, 212, 312 are stainless steel strands, so are generally flexible. This construction makes the electrodes 104, 204, 304 and hence the electrode leads 100, 200, 300 largely flexible.

The elongate elements 106, 206, 306 are hollow and one or more wires (not shown) extend along the inside of the elements 106, 206, 306 to provide electrical connection between the electrodes 104, 204, 304 and the connectors 108, 208, 308. Each connector 108, 208, 308 houses an electronically readable memory 124, 224, 324 and has four connector ports 114, 116, 118, 120; 214, 216, 218, 220; 314, 316, 318, 320. An anode connector port 114, 214, 314 and a cathode connector port 116, 216, 316 are used to apply electrical potential to the electrodes 104, 204, 304. A clock connector port 118, 218, 318 and data connector port 120, 220, 320 are used to transfer data between the electronically readable memory 124, 224, 324 of the lead 100, 200, 300 and a temporary electrical pulse generator 400, described in more detail below.

The elongate elements 106, 206, 306 are each between around 70 mm and 300 mm long and have a diameter of around one millimetre, e.g. in this embodiment approximately 1.25 mm, with the electrode arrays 102, 202, 302 extending along the length of the elements 106, 206, 306 for between around 70 mm to 140 mm. More specifically, each lead 100, 200, 300 has length L from its connector 108, 208, 308 to the end of the lead 100, 200, 300 distal to the connector 108, 208, 308; the electrodes 104, 204, 304 extend along the each lead 100, 200, 300 for an overall distance L_{E}; the electrodes 104, 204, 304 are spaced apart from one another by a distance L_{A}; and the individual contacts 110, 210, 310 extend along the leads 100, 200, 300 for a distance L_{C} and are spaced apart from one another by a distance L_{S}. More precise details of the leads 100, 200, 300 and the values of the lengths and distances L, L_{E}, L_{A}, L_{C} and L_{S} are given in Table 1 below.

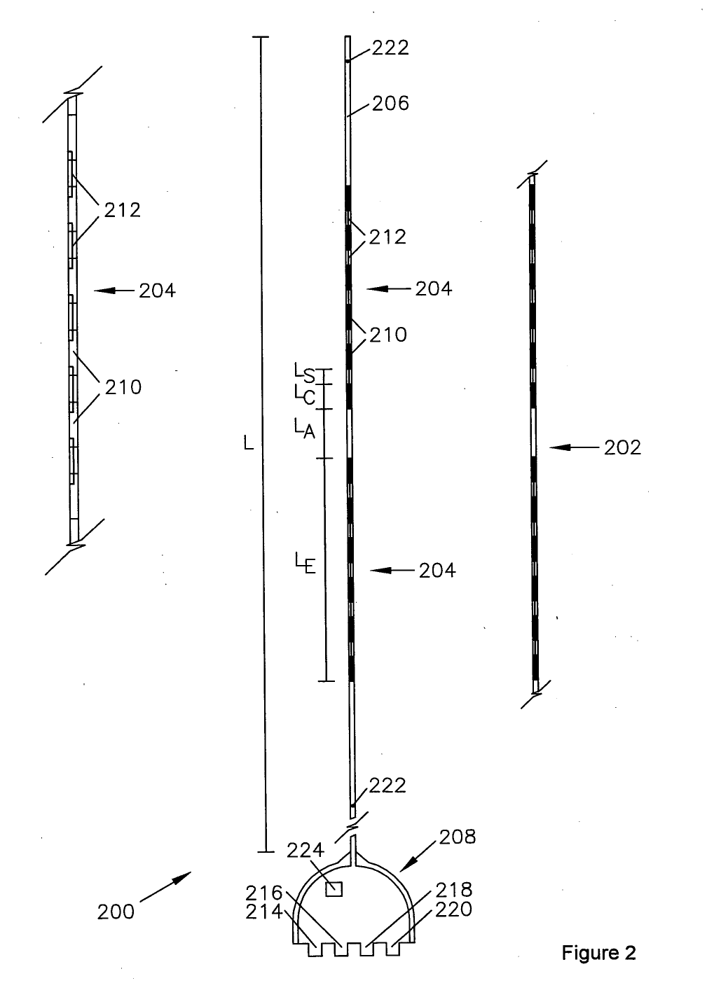

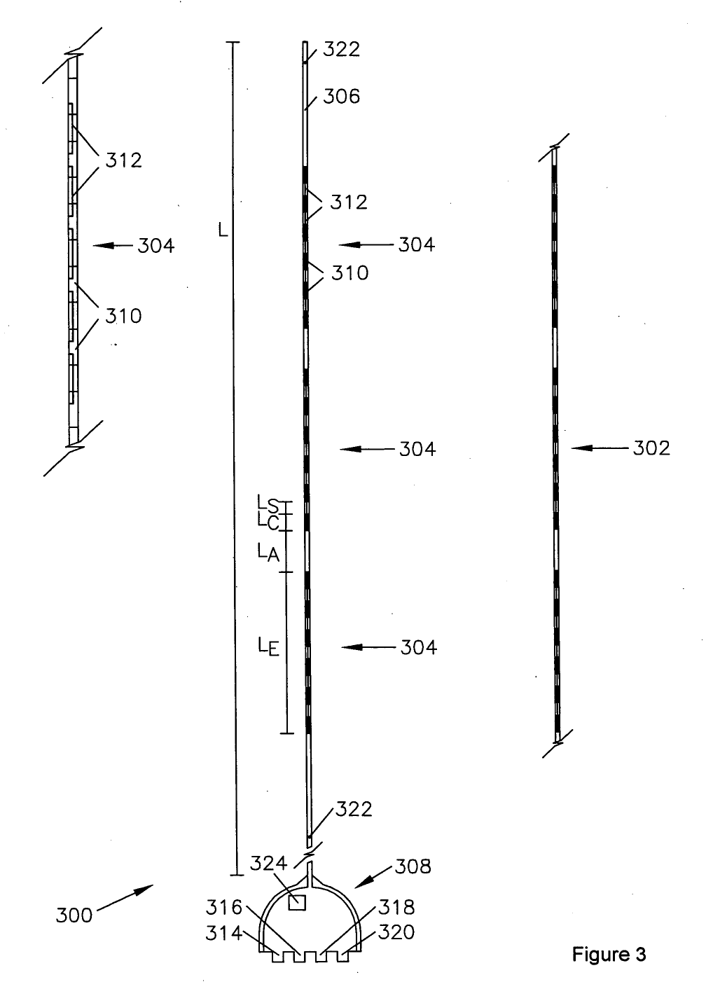

TABLE 1

| | Lead 100 | Lead 200 | Lead 300 |
|---|---|---|---|
| | (FIG. 1) | (FIG. 2) | (FIG. 3) |
| No. of electrodes 104, | 2 | 2 | 3 |
| 204, 304 | | | |
| No. of contacts 110, | 4 | 6 | 6 |
| 210, 310 per electrode | | | |
| 104, 204, 304 | | | |
| L (mm) | 163 | 195 | 232 |
| L_{E} (mm) | 29 | 45 | 39 |
| L_{A} (mm) | 10 | 10 | 10 |
| L_{C} (mm) | 5 | 4 | 4 |
| L_{S} (mm) | 3 | 3 | 3 |

Finally, a suture point 122, 222, 322 is provided close to each end of the elongate elements 106, 206, 306 for securing the leads 100, 200, 300 in a body. In these embodiments, the suture points 122, 222, 322 are each holes extending through the elongate elements 106, 206, 306.

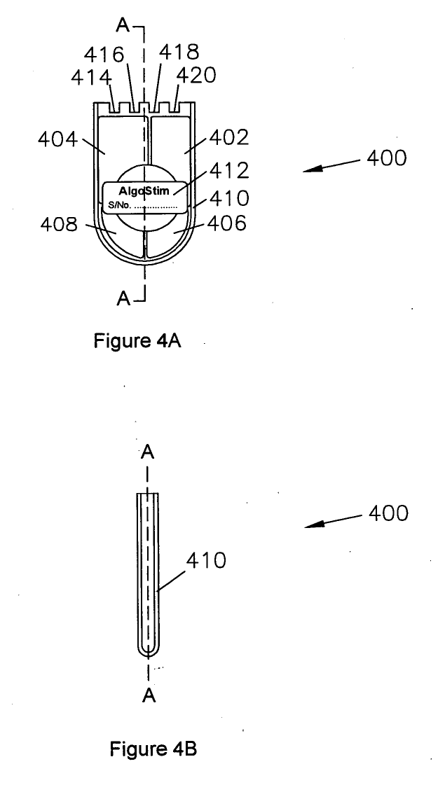

Referring to FIGS. 4A and 4B, a temporary electrical pulse generator 400, intended to be used outside the body, can be connected to the connector 108, 208, 308 of an electrode lead 100, 200, 300. The pulse generator 400 has electrical components including a power supply 402, a processor 404, a voltage conversion and current regulation unit 406 and a wireless communication device 408 mounted in a housing 410. In this embodiment, the power supply 402 is a lithium battery with a capacity of around 500 mAh; the processor 404 is a small conventional central processing unit (CPU) that can communicate with a switching circuit (not shown) and control the electrical current, voltage and waveform applied to the electrodes 104, 204, 304 of the leads 100, 200, 300; the voltage conversion and current regulation unit 406 is able to step the DC voltage of the power supply 402 to a DC voltage selected by the processor 404 and maintain a constant current supply from the power source 402; and the wireless communication device 408 is able to communicate with a control unit 700, a programming unit 800 and a wireless modem 1200 (described below), e.g. using Bluetooth® or Wi-Fi® communication standards. Also mounted in the pulse generator 400 is an ID tag 412 made from a radio opaque material and marked with a serial number and manufacturer identification in such a way that the serial number and manufacturer identification show up on X-ray images of the pulse generator 400.

In order to be able to mate with the electrode leads 100, 200, 300, the temporary pulse generator 400 has four connector ports 414, 416, 418, 420 for mating with the connector ports 114, 116, 118, 120; 214, 216, 218, 220; 314, 316, 318, 320 of the leads 100, 200, 300. An anode connector port 414 and a cathode connector port 414 are used to apply electrical potential to the electrodes 104, 204, 304 of the leads 100, 200, 300. A clock connector port 418 and data connector port 420 are used to transfer data between the electronically readable memory 124, 224, 324 of the lead 100, 200, 300 and the pulse generator 400.

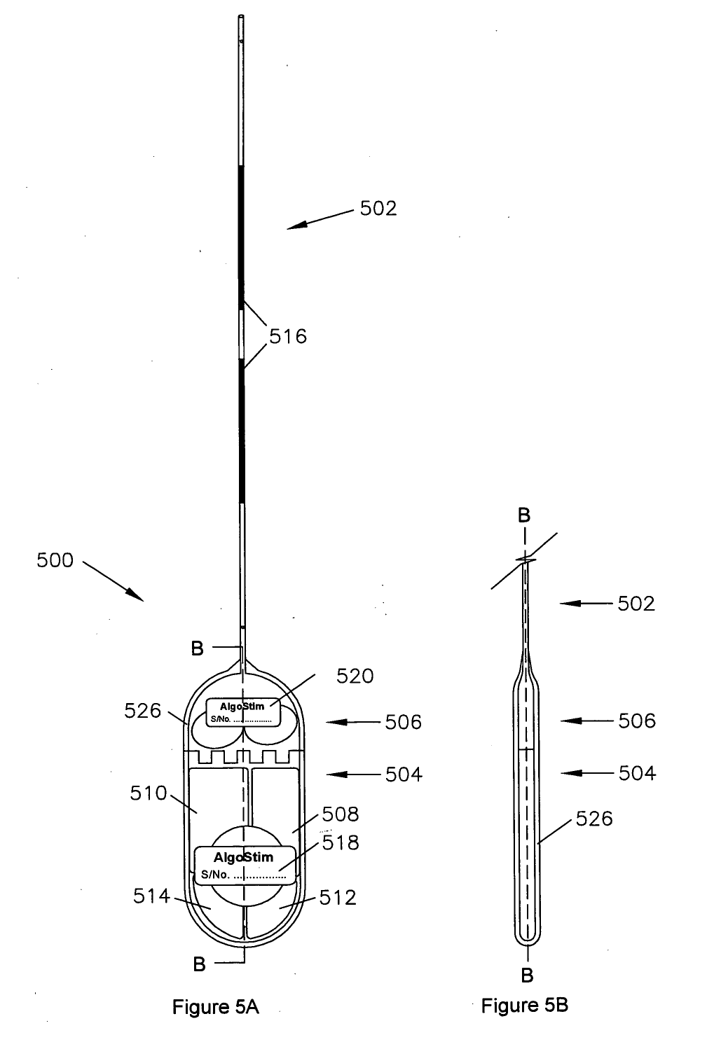

Referring to FIGS. 5A and 5B, an electrical nerve stimulation device 500 for insertion in the body comprises a permanent electrode lead 502 and a pulse generator 504. The pulse generator 504 is positioned at one end of the electrode lead 502 and mates with a connector 506 of the lead 502. The pulse generator 504 and the connector 506 of the lead 502 together form a unit substantially in the shape of a flat oval with curved edges. The dimensions of the unit, e.g. the combined dimensions of the pulse generator 504 and connector 506, are around 52 mm long, 23 mm wide and 5 mm thick.

The pulse generator 504 has electrical components including a power supply 508, a processor 510, a voltage conversion and current regulation unit 512 and a wireless communication device 514 mounted on a flexible circuit board. In this embodiment, the power supply 508 is a lithium battery with a capacity of around 500 mAh; the processor 510 is a small conventional central processing unit (CPU) that can communicate with a switching circuit (not shown) and control the electrical current, voltage and waveform applied to the electrodes 516 of the lead 502; the voltage conversion and current regulation unit 512 is able to step the DC voltage of the power supply 508 to a DC voltage selected by the processor 510 and maintain a constant current supply from the power source 508; and the wireless communication device 514 is able to communicate with the control unit 700, the programming unit 800 and the wireless modem 1200, e.g. using Bluetooth® or Wi-Fi® communication standards. Mounted in both the pulse generator 504 and the connector 506 of the lead 502 are ID tags 518, 520 made from a radio opaque material and marked with a serial number and manufacturer identification in such a way that the serial number and manufacturer identification show up on X-ray images of the device 500.

The lead 502 has the same components and construction as the temporary leads 100, 200, 300 described above, although in this embodiment the lead 502 does not have an electronically readable memory and the clock connector port and data connector port are redundant. The lead 502 is also shorter than the temporary leads 100, 200, 300, as it does not need to extend outside the body, but only to the pulse generator 504 located inside the body. More specifically, the lengths L for the leads 100, 200, 300 in Table 1 are reduced to 133 mm, 165 mm and 202 mm respectively and the distance from the unit formed by the pulse generator 504 and the connector 506 and the beginning of the electrode 516 closest to the unit is no more than around 5 cm.

The lead 502 and pulse generator are connected to one another during manufacture and sealed to one another. In this embodiment, this is achieved by moulding the housing around the connected lead 502 and pulse generator 504 from molten silicone. The silicone solidifies to form a housing 526 enclosing the device 500 from around the suture loop proximal to the pulse generator 504 toward and including the entire pulse generator 504.

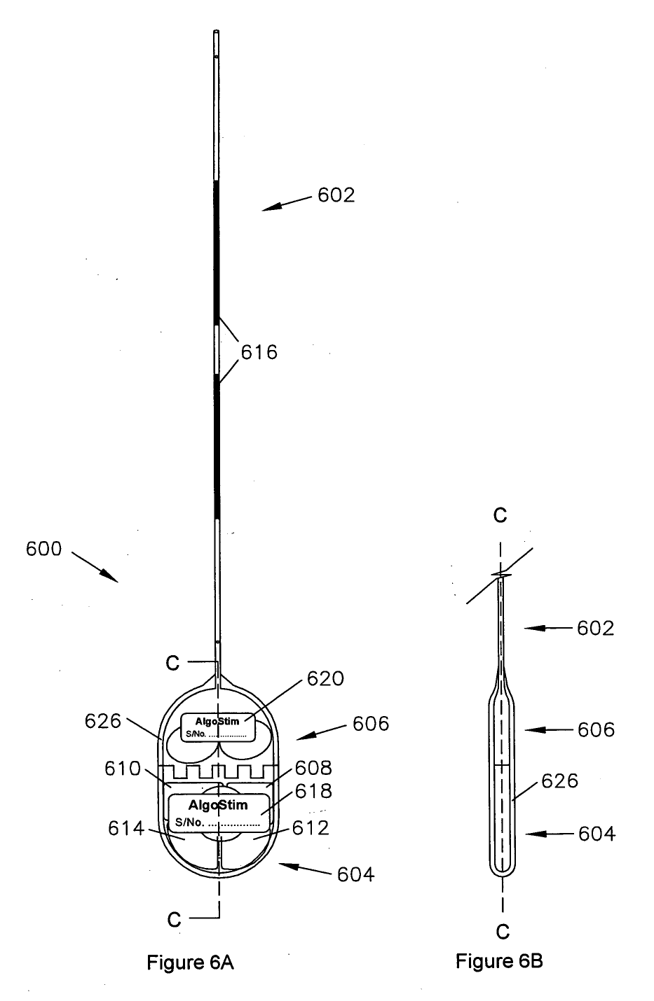

Referring to FIGS. 6A and 6B, in another embodiment, an electrical nerve stimulation device 600 smaller than the device 500 illustrated in FIGS. 5A and 5B also comprises an electrode lead 602 and a pulse generator 604. The components of the smaller device 600 are analogous to those of the larger device 500 and are labelled with corresponding reference numerals in the drawings. However, the power supply 608 of the smaller device 600 comprises a lithium battery having a smaller capacity of around 40 mAh. Along with careful packaging of the components of the pulse generator 604, this smaller capacity enables the connector 606 and pulse generator 604 of the smaller device 600 to form a smaller unit substantially in the shape of a flat oval with curved edges. In this embodiment, the dimensions of the unit, e.g. the combined dimensions of the pulse generator 604 and connector 108, 208 308, are around 36 mm long, 22 mm wide and 5 mm thick.

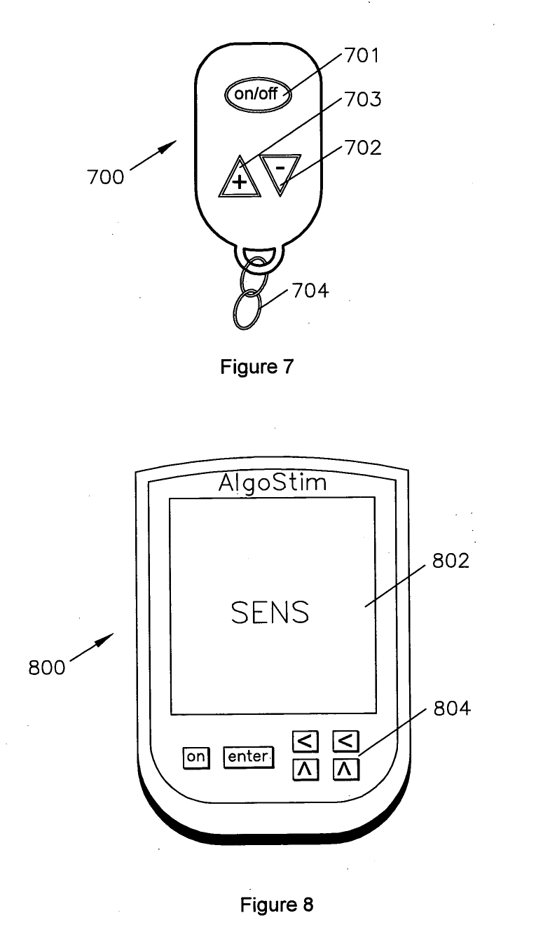

Referring to FIG. 7, a control unit 700 comprises a wireless communication device, which in this embodiment comprises a key fob or such like. The control unit 700 has radio transmitter (not shown) for communicating with the wireless communication device 408; 514; 614 of the pulse generator 400; 504; 604. An on/off button 701 on the control unit 700 can be operated by a patient to cause the transmitter to transmit signals to the wireless communication device 408; 514; 614 to turn the pulse generator 400; 504; 604 on or off. Similarly, amplitude control buttons 702; 703 on the control unit 700 can be operated by a patient to cause the transmitter to transmit signals to the wireless communication device 408; 514; 614 to increase or decrease the current or voltage of the electrical pulses output by the pulse generator 400; 504; 604.

Referring to FIG. 8, a programming unit 800 is provided for remotely programming the pulse generators 400, 504, 604. The unit 800 comprises a screen 802 and a user input buttons 804 and houses a processor, memory and wireless communication device (not shown), similar to those of a conventional personal digital assistant (PDA). The programming unit 800 can receive commands from a user via the buttons 804 and display information to the user on the screen 802. In addition, it can wirelessly transmit commands to and receive information from the wireless communication devices 408, 514, 614 of the pulse generators 400, 504, 604.

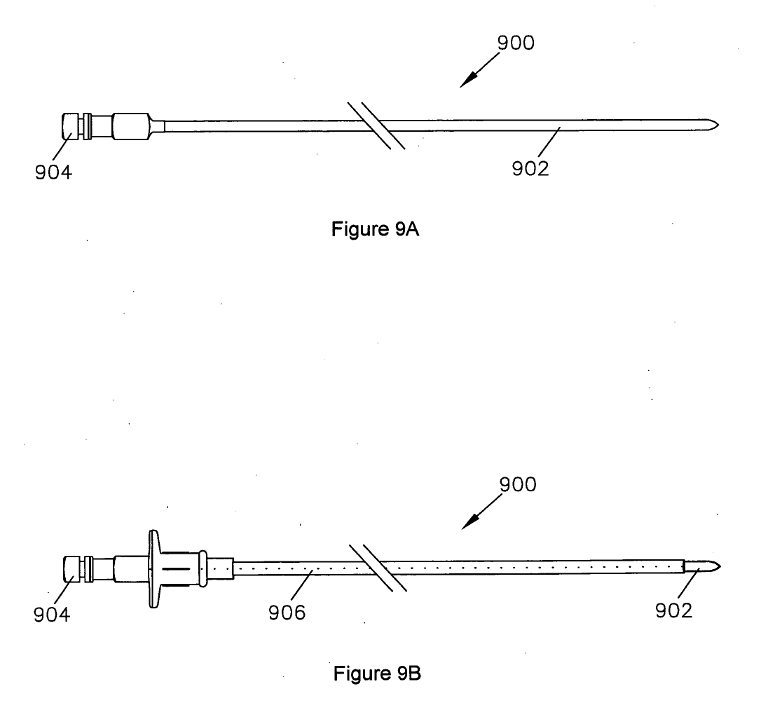

Referring to FIGS. 9A and 9B, an introducing instrument 900 for introducing the leads 100, 200, 300, 502, 602 into the body comprises a needle 902 with a length around the same as that of the corresponding lead 100, 200, 300, 502, 602 and a manipulator 904 at one end of the needle 902. In this embodiment, the needle 902 is a standard 14 gauge Touhy needle (which is a standard hollow needle). As can be seen in FIG. 9B, the instrument 900 is fitted with a sheath, referred to as a peel sheath 906, extending over the needle 902. After insertion of the instrument 900 into the body, the needle 902 can be withdrawn leaving the peel sheath 906 in place in the body. The lead 100, 200, 300, 502, 602 can then be inserted at a desired position in the body by passing it into the peel sheath 906. Once the lead 100, 200, 300, 502, 602 is in place, the peel sheath 906 can be removed by withdrawing it from the body. The peel sheath 906 is too narrow to pass over the connector 108, 208, 308, 506, 606 and pulse generators 504, 604, but can be torn as it is withdrawn so that it peels away from the lead 100, 200, 300, 502, 602. So, in this embodiment, the peel sheath 906 is made from a thin plastics film or such like to allow tearing.

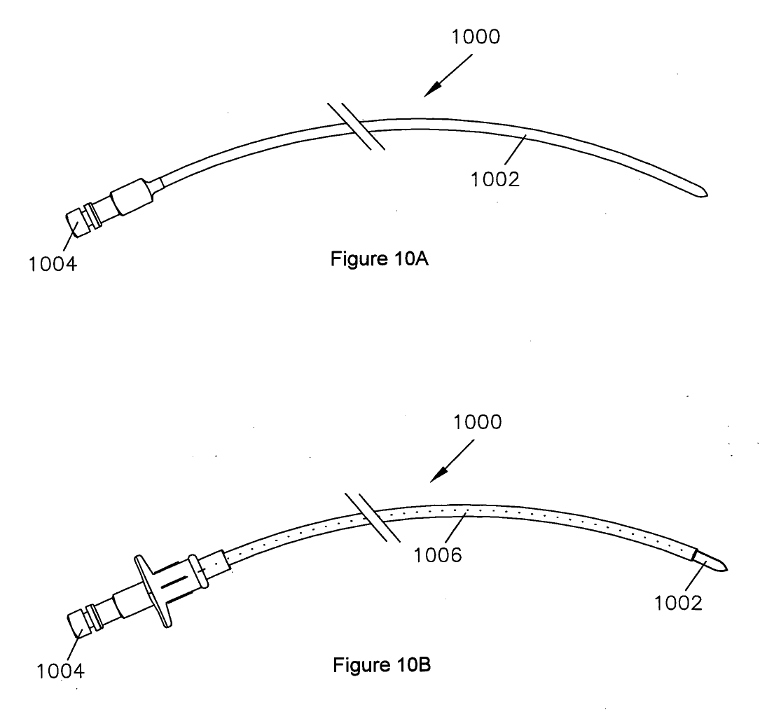

The needle 902 of the instrument 900 illustrated in FIGS. 9A and 9B is substantially straight. However, in another embodiment, illustrated in FIGS. 10A and 10B, an introducing instrument 1000 has a curved needle 1002. This is suitable for introducing the leads 100, 200, 300, 502, 602 into curved sites in a body, such as under a breast. In other respects, the manipulator 1004 and peel sheath 1006 of the instrument 1000 of this embodiment are similar to those of the previous embodiment.

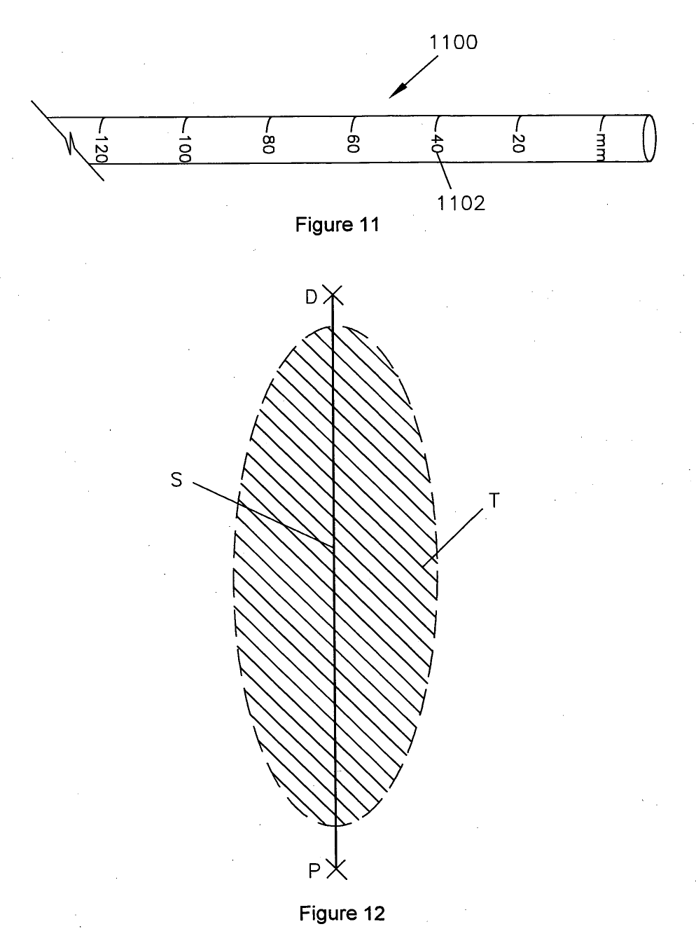

Referring to FIG. 11, a marker 1100 comprises a flexible elongate element having length markings 1102 along its length. At least the length markings 1102 are radio opaque so that they show up in X-ray images, e.g. during fluoroscopy. In this embodiment, the marker 1100 is made of silicone. Of course, other durable and flexible materials may be used in other embodiments as desired.

In order to treat a patient, it is first necessary to identify a target treatment area. This may be relatively clear, e.g. when it is intended to alleviate pain at the site of a wound or such like, as may be the case following surgery, or to treat specific pain areas, such as post inguinal hernia repair pain, penile/scrotal/testicular pain or vulvadynia. However, it is generally necessary to carry out a test to accurately identify the target treatment area. One such test is known as the “pin prick and cotton wool method”. This involves mapping the area of greatest allodynia by stimulating the surface of the skin with cotton wool in both an area of suspected allodynia and another area for comparison and monitoring patient response. Similarly, it involves stimulating an area of suspected hyperalgesia by probing with a pin pricks and monitoring patient response. The target treatment area is identified as the area of allodynia and/or hyperalgesia, and preferably the target treatment area is identified as the area of greatest allodynia and/or hyperalgesia. Those areas having the greatest allodynia and/or hyperalgesia are identified as those areas having the greatest pain sensation.

Once the target area has been identified, the first step is to insert the introducing instrument 900, 1000. Either the straight introducing instrument 900 or the curved introducing instrument 1000 is used, depending on the shape of the target area. Referring to FIG. 12, in an illustrated embodiment, a basically straight elongate target area T has been identified. The straight introducing instrument 900 is therefore selected for insertion of one of the electrode leads 100, 200, 300. In this example, a temporary electrode lead 100, 200, 300 is first inserted into the target area T. One of the leads 100, 200, 300 is selected based on the size of the target area T and the treatment regime to be administered. The selected lead 100, 200, 300 should be inserted along the central or major axis of the target area T and the selected lead 100, 200, 300 is laid on the skin over appropriate site S in the target treatment area T. The desired position P of the suture point 122, 222, 322 proximal to the connector 108, 208, 308 and the desired position D of the suture point 122, 222, 322 distal to the connector 108, 208, 308 are then marked on the skin. Once the lead 100, 200, 300 has been moved away, an incision is made at each of these positions P, D. The incisions are of sufficient size to allow the lead 100, 200, 300 to be secured by sutures once in position. The needle 902 of the introducing instrument 900 (with peel sheath 906 in place) is then inserted through the skin (e.g. “percutaneously”) and tunnelled subcutaneously along the desired site S of the lead 100, 200, 300 in the target area T between the incisions at the two positions P, D. The needle 902 typically extends through the fatty tissue directly underneath the skin at around 5 mm below the surface of the skin.

Once the needle 902 has been inserted at the desired site S, it is withdrawn leaving the peel sheath 906 in place. The selected lead 100, 200, 300 is then inserted into the peel sheath 906 and consequently the site S, with the connector 108, 208, 308 left external to the body close to the position P. So, the lead 100, 200, 300 is positioned along the site S and extends between the incisions at the positions P, D. The peel sheath 906 is then withdrawn from the body by pulling it out of the insertion point of the lead 100, 200, 300 and tearing it away from the lead 100, 200, 300 and to open it over the connector 108, 208, 308 of the lead 100, 200, 300.

Generally speaking, as the lead 100, 200, 300 has been carefully inserted between the two incisions, there is no need for fluoroscopic confirmation of the location of the lead 100, 200, 300. However, if confirmation is required, the marker 1100 is positioned on the skin over the site S in the target area T and the location of the lead 100, 200, 300 is verified by fluoroscopy. In particular, the axial position of the lead 100, 200, 300 can be adjusted during fluoroscopy to position the electrodes 104, 204, 304 at the desired location along the site S.

Once the lead 100, 200, 300 has been inserted, it is anchored to body tissue at the desired positions P, D using sutures attached to the suture points 122, 222, 322. The incisions made at the points P, D are then closed using appropriate sutures and the temporary pulse generator 400 is connected to the connector 108, 208, 308 of the lead 100, 200, 300 (which protrudes from the insertion point of the lead 100, 200, 300). The pulse generator 400 can then be secured to the patient's body using a dressings or such like and the programming unit 800 used to program the pulse generator 400.

First, the pulse generator 400 accesses the memory of the lead 100, 200, 300 to retrieve a product code and transmits the product code to the programming unit 800. The programming unit 800 uses the retrieved product code to identify the type of lead 100, 200, 300 that has been implanted. This enables the programming unit 800 to retrieve a list of treatment regimes suitable for use with the particular type of lead 100, 200, 300 from its memory. This list is then displayed on the screen 802 of the programming unit 800.

The treatment regimes all comprise a series of electrical pulses, each pulse having duration between around 100 μs and 500 μs, and typically around 200 μs. A range of frequencies are available, between around 1 Hz and 60 Hz, but the frequency is usually toward the lower end of this range, e.g. 2 Hz. The pulse generator can operate in either a constant current mode or a constant voltage mode. In the constant current mode, the current is fixed at a value less than around 5 mA, typically between around 1 mA and 3 mA. The patient is then able to vary the voltage of the electrical pulses using the amplitude control buttons 702, 703 of the controller 700 to adjust the level of pain relief as desired. However, the voltage does not usually exceed 200 V. In the constant voltage mode, the voltage is fixed at a value less than around 220 V, typically 200V. The patient is then able to vary the current of the electrical pulses using the amplitude control buttons 702, 703 of the controller 700 to adjust the level of pain relief as desired. Whilst the current might be varied between around 0 mA and 5 mA, it is typically set between around 1 mA and 3 mA.

In this embodiment, either a square waveform or an H-wave bi-polar exponentially decaying waveform is used. For example, as shown in FIG. 14A, a square voltage pulse having duration 200 μs and amplitude 200V is applied at 60 Hz. In another example, as shown in FIG. 14B, a decaying pulse of duration 200 μs and maximum amplitude 200V is applied with alternating polarity at 2 Hz. Different waveforms, frequencies and fixed currents or voltages can be selected from the list of treatment regimes according to a patient's response to the treatment.

So, an appropriate treatment regime can be selected by a physician from the list displayed on the screen 802 using buttons 804 of the programming unit 800. Once the treatment regime has been selected, it is transmitted by the programming unit 800 to the pulse generator 400. At the same time, a patient ID, implantation date and such like are sent to the pulse generator 400 and stored in a memory (not shown) of the pulse generator 400 or of the lead 100, 200, 300.

After the treatment regime has been completed, a physician can assess the effectiveness of the treatment and vary the treatment regime appropriately using the programming unit 800. If the treatment is effective, e.g. a patient experiences a 50% or more reduction in pain, it may be decided to implant a more permanent device. In the event that it is decided to implant a permanent deyice, the temporary lead 100, 200, 300 is removed. A fully implantable electrical nerve stimulation device 500, 600 is then selected for implantation, for example suitable for replicating the desired treatment regime over a period of a year or more.

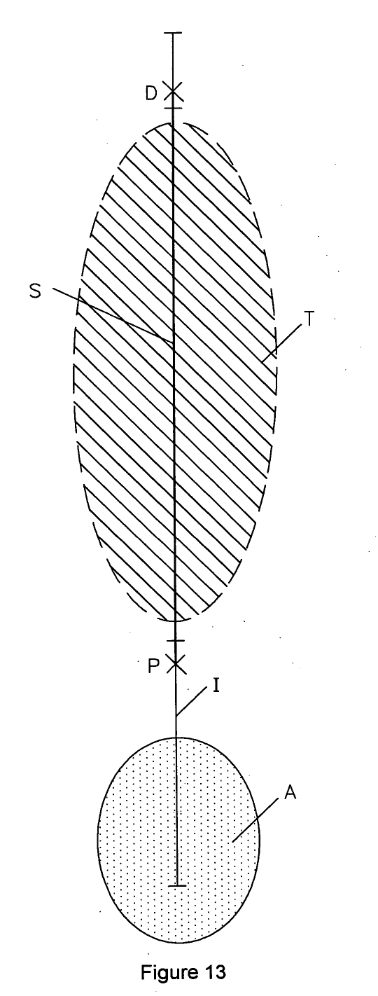

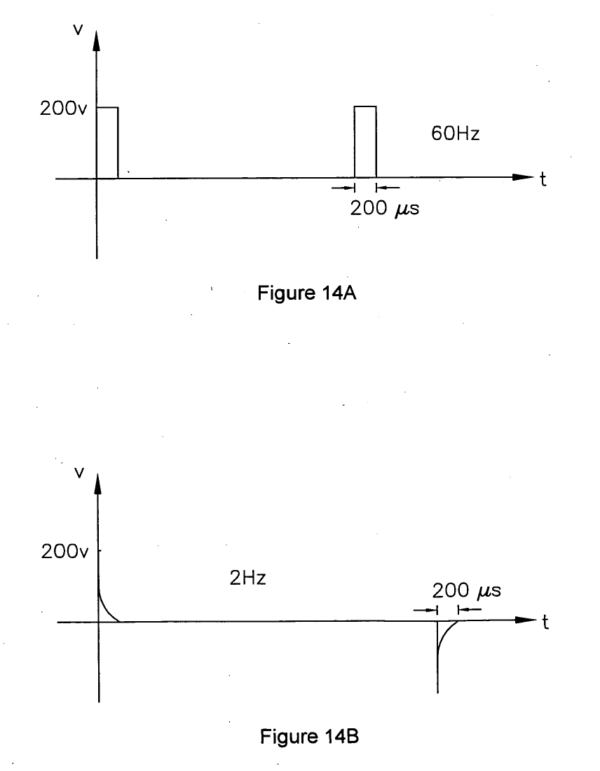

The lead 502, 602 of the selected electrical nerve stimulation device 500, 600 is implanted in the site S of the temporary lead 100, 200, 300 using the same method by which the temporary lead 100, 200, 300 was originally inserted. In other words, the lead 502, 602 is implanted using the introducing implement 900, 1000 and sutured at positions P, D. Referring to FIG. 13, once the lead 502, 602 of the device 500, 600 has been inserted, the incision at the point P is extended along path I away from the site S of the lead 502, 602. This incision can be used by a surgeon to open up a pocket under the patient's skin in area A for receiving the pulse generator 504, 604 of the device 500, 600. The pulse generator 504, 604 is inserted in the pocket no more than around 2 cm and typically around 5 mm below the skin surface, taking care not to twist or bend the lead 502, 602 and ensuring that the ID-tags 518, 520, 618, 620 face the skin. The incision along path I is then closed with sutures in a conventional manner so that the device 500, 600 is fully implanted.

The implanted device 500, 600 is able to automatically deliver electrical pulses to the target area T via the lead 502, 602 in accordance with a pre-selected treatment regime. It can also be reprogrammed using the programming unit 800 and the control unit 700 can be used to turn the device on or off and increase and decrease the current or voltage of the electrical pulses.

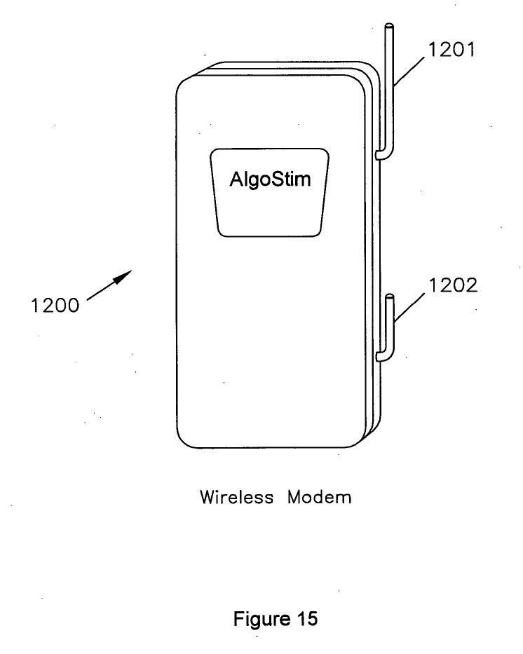

In addition, a wireless modem 1200 can be provided to a patient for use in their home or at any other location remote from the physician or hospital responsible for the treatment. Referring to FIG. 15, the modem 1200 has two aerials 1201 and 1202. Using the first aerial 1201, the modem communicates with a telephone network, e.g. via a patient's home telephone using the digital enhanced cordless telecommunications (DECT) standard, or with a mobile telephone network, e.g. using the general packet radio service (GRPS) standard. Using the second aerial 1202, the modem communicates with the wireless communication device 408, 514, 614 of the pulse generators 400, 504, 604. So, the modem can establish a communications link between a physician operated communication device over a telephone network and the pulse generators 400, 504, 604, allowing remote reprogramming of the pulse generators 400, 504, 604 by a physician.

In this embodiment, the device 500, 600 can be programmed to turn itself off automatically after a predetermined time to save battery power or to operate continuously, depending on the treatment needs of the patient. Regardless, the battery will inevitably expire after a given time, typically between two and seven years. When this happens, the device 500, 600 is removed and can be replaced is desired.

The invention can be used to treat neuropathic pain in virtually any location of the body and arising from a multitude of different causes. Some examples are listed below.

Post Mastectomy Pain

The most commonly cited theory of chronic postoperative pain in breast cancer patients is the intentional sacrificing of the intercostobrachial nerves. These sensory nerves exit through the muscles of the chest wall, and provide sensation predominantly to the shoulder and upper arm. Because these nerves usually run through the packet of lymph nodes in the armpit, they are commonly cut by the surgeon in the process of removing the lymph nodes. Symptoms are described as burning, tingling, itching, or frank lancinating pain. In a small percentage of patients, chronic pain results, and the painful symptoms persist. The symptoms may be present almost continually, or they may occur in response to changes in physical activity or temperature. They may also be exacerbated by physical contact with the affected area i.e. the surgical scar, chest wall, breast, axilla and or ipsilateral upper extremity.

The incidence of chronic pain syndromes following breast cancer treatment has been estimated to occur in 20-25% patients undergoing axillary (armpit) dissection, with or without mastectomy. Additional factors linked to breast cancer-associated chronic pain syndromes include polyneuropathies caused by chemotherapy and radiation therapy, which may be additive to impairments caused by surgery.

Such post mastectomy pain can be treated by inserting the electrode lead 100, 200, 300, 502, 602 in the area of greatest hyperalgesia.

Lymphedema (Post Mastectomy)

Whenever the normal drainage pattern in the lymph nodes is disturbed or damaged (often during surgery to remove the lymph nodes during mastectomy), swelling of the arm may occur. Radiation and chemotherapy may also cause swelling of the arm. This swelling of the arm, caused by an abnormal collection of too much fluid, is called lymphedema.

When the lymph nodes under the arm have been removed, a woman is at higher risk of lymphedema. Lymphedema may occur immediately following surgery, or months or years later. Not every woman who has a mastectomy will experience lymphedema.

There are several types of lymphedema. The acute, temporary, and mild type of lymphedema occurs within a few days after surgery and usually lasts a short period of time. The acute and more painful type of lymphedema can occur about 4 to 6 weeks following surgery. However, the most common type of lymphedema is slow and painless and may occur 18 to 24 months after surgery.

The main symptom of lymphedema is swelling of the affected arm. The degree of swelling may vary. Some people may experience severe swelling (edema)—with the affected arm being several inches larger than the other arm. Others will experience a milder form of edema—with the affected arm being slightly larger than the other arm.

In addition to swelling of the affected arm, the most common symptoms of lymphedema include feeling of fullness or tightness in the affected arm, aching or pain in the affected arm, swelling in the hand (may be evidenced by rings that no longer fit) and weakness in the affected arm. However, each individual may experience symptoms differently.

The pain and swelling of lymphedhema can be alleviated using the invention by implanting the electrode lead 100, 200, 300, 502, 602 in the affected arm, again in the area of greatest hyperalgesia.

Neuropathic Chest Wall Pain

Neuropathic chest wall pain is chronic pain that occurs following surgery or resulting from a medical condition such as an infection, cystic fibrosis or such like and can cause respiratory function reduction. It can be treated using the invention by implanting the electrode lead 100, 200, 300, 502, 602 in the area, e.g. of the chest wall, at which the patient experiences greatest hyperalgesia.

Chronic Post Surgical Pain

Chronic post surgical pain is pain that develops after a surgical procedure and is still present more than 2 months after surgery. It can be treated using the invention by implanting the electrode lead 100, 200, 300, 502, 602 in the area, e.g. the surgical wound, at which the patient experiences greatest hyperalgesia.

Complex Regional Pain Syndrome (CRPS)

This is a post traumatic sensory or mixed nerve neuropathic pain that can follow trauma/surgery/myocardial infarct. It is a chronic pain condition and a patient with CRPS has pain as well as changes in blood flow, sweating, and swelling in the painful area. Sometimes the condition leads to changes in the skin, bones and other tissues. It may also become hard for a patient with CRPS to move the painful body part. The patient's arms or legs are usually involved, but CRPS may affect any part of the body, such as the face or trunk. In some patients, many different areas of the body are affected. CRPS can be progressive. CPRS usually develops after an injury to the skin, bone, joints or tissue. This type of CRPS has been called reflex sympathetic dystrophy. CRPS can also develop after any type of injury to major nerves. This type has been called causalgia. The injury that leads to CRPS may be only minor, and sometimes a patient cannot remember any injury or event that caused CRPS to start.

CPRS can be treated using SCS. However, some CPRS patients successfully treated by SCS still experience discrete areas of hyperalgesia or allodynia. These areas can be treated using the invention by implanting the electrode lead 100, 200, 300, 502, 602 in the area, e.g. of the affected limb, at which the patient experiences greatest hyperalgesia or allodynia.

Neuropathic Head, Neck and Facial Pain

This can occur when there is an area of hyperalgesia on head or neck and can be treated using the invention by implanting the electrode lead 100, 200, 300, 502, 602 in the area, e.g. of the head neck or face, at which the patient experiences greatest hyperalgesia.

Neuropathic Foot Pain

This can occur when there is an area of pain in a distribution of a sensory nerve in the foot. Some patients get significant pain relief using SCS. However, some of these patients still experience discreet areas of hyperalgesia or allodynia and these can be treated using the invention by implanting the electrode lead 100, 200, 300, 502, 602 in the area, e.g. of the foot, at which the patient experiences greatest hyperalgesia or allodynia. In patients where the pain only in a discreet area, SCS can be avoided and the discreet area treated using the invention.

Penile/Scrotal/Testicular Pain

This can occur when there is focal neuropathic pain at the penis, scrotum or testicles and can be treated using the invention by implanting the electrode lead 100, 200, 300, 502, 602 in the inguinal canal.

Post Inguinal Hernia Repair Pain

This can occur when there is focal neuropathic pain at the site of an inguinal hernia repair and can be treated using the invention by implanting the electrode lead 100, 200, 300, 502, 602 at the site or in the inguinal canal.

Neuropathic Abdominal Wall Pain

It has been proposed that cutaneous nerve roots can become injured where they pass through the abdominal wall, perhaps by the stretching or compression of the nerve root along its course through the abdominal fascia. In some instances, a tight belt or other poorly fitted clothing can cause nerve root irritation, especially in physically unfit persons with protuberant abdomens. Pain also can occur in or around the abdominal wall where muscles insert on bones or cartilage. For example, the pain can occur where the rectus abdominis muscles insert on the lower ribs or where the lower ribs connect through cartilage. The xiphoid cartilage is sometimes a specific focus of pain.

Most commonly, abdominal wall pain is related to cutaneous nerve root irritation or myofascial irritation. The pain can also result from structural conditions, such as localized endometriosis or rectus sheath haematoma, or from incisional or other abdominal wall hernias. If hernia or structural disease is excluded, injection of a local anaesthetic with or without a corticosteroid into the pain trigger point can be diagnostic and therapeutic.

Pain that is the same or increased when the abdominal wall is tensed generally indicates an origin in the abdominal wall. The mechanism for the pain may involve the development of an area of hyperalgesia as a result of myofascial stretch injury.

Neuropathic abdominal wall pain can be treated using the invention by implanting the electrode lead 100, 200, 300, 502, 602 in the area at which the patient experiences greatest hyperalgesia. A tender trigger point in the abdominal wall is frequently no more than 1 or 2 cm in diameter. However, it is not unusual for the pain to spread over a wide area or to be referred. For instance, pressing on a tender trigger point in the right upper quadrant (nerve root T7) can refer pain to the angle of the scapula. Patients are often so preoccupied with the large area of pain spread that they do not realize the area of tenderness is extremely localized and superficial.

Neuropathic Failed Back Surgery Syndrome (FBSS)

This can occur following back surgery, e.g. for post spinal fusion or discectomy. Many patients get significant pain relief from SCS, although there are instances where discrete areas of hyperalgesia or allodynia persist. These discrete areas of hyperalgesia or allodynia can be treated using the invention by implanting the electrode lead 100, 200, 300, 502, 602 in the area at which the patient experiences greatest hyperalgesia or allodynia. In other words SCS and the invention are used simultaneously.

Angina

Angina that is no longer treatable by surgical or medical interventions is called refractory angina (previously known as brittle or end-stage angina). This diagnosis is made by a cardiologist, cardiac surgeon or both. Classed as a chronic pain syndrome it has profoundly damaging effects on the quality of life of the individual sufferer, their family and friends. Some patients find relief using SCS, although discrete areas of hyperalgesia or allodynia can remain. These discrete areas of hyperalgesia or allodynia can be treated using the invention by implanting the electrode lead 100, 200, 300, 502, 602 in the area at which the patient experiences greatest hyperalgesia or allodynia. In other words SCS and the invention are used simultaneously.

Migraine

Migraines are recurring intense headaches preceded by a sensory warning sign (aura), such as flashes of light, blind spots or tingling in your arm or leg. Migraines are also often accompanied by other symptoms, such as nausea, vomiting and extreme sensitivity to light and sound. Migraine pain can be excruciating and may incapacitate the sufferer for hours or even days.

The invention can be used to treat migraine by implanting the electrode lead 100, 200, 300, 502, 602 in the area of greatest hyperalgesia, e.g. in the occipital scalp.

Post Traumatic Cervical Neuropathic Pain

This comprises cervical neuropathic pain in an area of discrete nerve distribution and can be treated using the invention by implanting the electrode in the area of greatest hyperalgesia, e.g. at the cervix.

Vulvadynia

Vulvadynia (or Vestibulitis) is pain or discomfort of the female genitalia or surrounding area. Complaints may be of pain, burning, stinging, irritation, itching, inflammation or rawness. The discomfort can be constant or intermittent. Some women will only have pain when pressure is applied to the area surrounding the entrance of the vagina or the vestibule area. It can be caused by trauma, surgery or child birth for example.

Vulvadynia can be treated using the invention by implanting the electrode 100, 200, 300, 502, 302 in the inguinal canal.

Coccydynia

Pain in the area of the coccyx (tailbone) is called coccydynia or coccygodynia. Coccydynia can be anything from discomfort to acute pain, varying between people and varying with time in any individual. The name describes a pattern of symptoms (pain brought on or aggravated by sitting), so it is really a collection of conditions which can have different causes and need different treatments.

Coccydynia can follow after falls, childbirth, repetitive strain or surgery. In some cases the cause is unknown. The pain can disappear by itself or with treatment, or it can continue for years, and may get worse. It is five times more common in women than men, probably because the female pelvis leaves the coccyx more exposed. It appears that in most cases the pain is caused by an unstable coccyx, which causes chronic inflammation.

Coccydynia can be treated using the invention by implanting the electrode lead 100, 200, 300, 502, 602 in the area of greatest hyperalgesia.

The described embodiments of the invention are only examples of how the invention may be implemented. Modifications, variations and changes to the described embodiments will occur to those having appropriate skills and knowledge. These modifications, variations and changes may be made without departure from the spirit and scope of the invention defined in the claims and its equivalents.

1. An electrical nerve stimulation device, the device comprising:
an electrode lead having electrodes disposed along its length;
an electrical pulse generator connected to the electrode lead for applying electrical pulses to the electrodes; and
a fluid impermeable housing enclosing the connection between the lead and the pulse generator so that the device is a single unit.

2. The electrical nerve stimulation device of claim 1, wherein the fluid impermeable housing encloses the pulse generator.

3. The electrical nerve stimulation device of claim 1, wherein the fluid impermeable housing is silicone.

4. The electrical nerve stimulation device of claim 1, wherein the fluid impermeable housing is silicone.

5. The electrical nerve stimulation device of claim 1, wherein the distance along the lead between the pulse generator and the electrode closest to the pulse generator is less than around 5 cm.

6. The electrical nerve stimulation device of claim 1, comprising a housing containing the pulse generator, which housing is substantially flat.

7. The electrical nerve stimulation device of claim 1, wherein the pulse generator is substantially flexible.

8. The electrical nerve stimulation device of claim 1, wherein the electrical pulse generator comprises a flexible circuit board on which components of the pulse generator are mounted.

9. The electrical nerve stimulation device of claim 1, wherein the electrode lead has an electronically readable memory for storing data.

10. The electrical nerve stimulation device of claim 1, wherein the electrodes each comprise a group of electrical contacts.

11. An electrical nerve stimulation device that is implantable in a human or animal body, the device comprising:
an electrode lead having electrodes disposed along its length; and
an electrical pulse generator connected to the electrode lead for applying electrical pulses to the electrodes,
wherein the distance along the lead between the pulse generator and the electrode closest to the pulse generator is less than around 5 cm.

12. The electrical nerve stimulation device of claim 11, comprising a housing containing the pulse generator, which housing is substantially flat.

13. The electrical nerve stimulation device of claim 11, wherein the pulse generator is substantially flexible.

14. The electrical nerve stimulation device of claim 11, wherein the electrical pulse generator comprises a flexible circuit board on which components of the pulse generator are mounted.

15. The electrical nerve stimulation device of claim 11, wherein the electrode lead has an electronically readable memory for storing data.

16. The electrical nerve stimulation device of claim 11, wherein the electrodes each comprise a group of electrical contacts.

17. An electrical nerve stimulation device, the device comprising:
an electrode lead having electrodes disposed along its length;
an electrical pulse generator connected to the electrode lead for applying electrical pulses to the electrodes; and
a housing containing the pulse generator, which housing is substantially flat.

18. The electrical nerve stimulation device of claim 17, wherein the housing is less than around 7 mm thick.

19. The electrical nerve stimulation device of claim 17, wherein the housing is less than around 5 mm thick.

20. The electrical nerve stimulation device of claim 17, wherein the pulse generator is substantially flexible.

21. The electrical nerve stimulation device of claim 17, wherein the electrical pulse generator comprises a flexible circuit board on which components of the pulse generator are mounted.

22. The electrical nerve stimulation device of claim 17, wherein the electrode lead has an electronically readable memory for storing data.

23. The electrical nerve stimulation device of claim 17, wherein the electrodes each comprise a group of electrical contacts.

24. An electrical nerve stimulation device, the device comprising:
an electrode lead having electrodes disposed along its length; and
an electrical pulse generator connected to the electrode lead for applying electrical pulses to the electrodes,
wherein the pulse generator is substantially flexible.

25. The electrical nerve stimulation device of claim 24, wherein the electrical pulse generator comprises a flexible circuit board on which components of the pulse generator are mounted.

26. The electrical nerve stimulation device of claim 24, wherein the electrode lead has an electronically readable memory for storing data.

27. The electrical nerve stimulation device of claim 24, wherein the electrodes each comprise a group of electrical contacts.

28. An electrical nerve stimulation device, the device comprising:
an electrode lead having electrodes disposed along its length; and
an electrical pulse generator connectable to the electrode lead for applying electrical pulses to the electrodes,
wherein the electrode lead has an electronically readable memory for storing data.

29. The electrical nerve stimulation device of claim 28, wherein the data represents information about the lead.

30. The electrical nerve stimulation device of claim 28, wherein the memory is a read-only memory.

31. The electrical nerve stimulation device of claim 28, wherein the electrodes each comprise a group of electrical contacts.

32. An electrical nerve stimulation device, the device comprising:
an electrode lead having electrodes disposed along its length; and
an electrical pulse generator connectable to the electrode lead for applying electrical pulses to the electrodes,
wherein the electrodes each comprise a group of electrical contacts.

33. The electrical nerve stimulation device of claim 32, wherein each electrode comprises between 2 and 10 electrical contacts.

34. The electrical nerve stimulation device of claim 32, wherein the electrical contacts each extend for between around 2 mm and 5 mm along the length of the electrode lead.

35. The electrical nerve stimulation device of claim 32, wherein the electrical contacts are spaced apart along the length of the electrode lead by around 2 mm or more.

36. An electrode lead for electrical nerve stimulation, the lead having electrodes disposed along its length and an electronically readable memory for storing data.

37. The electrode lead of claim 36, wherein the data represents information about the lead.

38. The electrode lead of claim 36, wherein the memory is a read-only memory.

39. The electrode lead of claim 36, the lead having electrodes that each comprise a group of electrical contacts.

40. An electrode lead for electrical nerve stimulation, the lead having electrodes that each comprise a group of separate electrical contacts.

41. The electrode lead of claim 40, wherein each electrode comprises between 2 and 10 electrical contacts.

42. The electrode lead of claim 40, wherein the electrical contacts each extend for between around 2 mm and 5 mm along the length of the electrode lead.

43. The electrode lead of claims 40, wherein the electrical contacts are spaced apart along the length of the electrode lead by around 2 mm or more.

44. A method of manufacturing an electrical nerve stimulation device, the method comprising:
connecting an electrode lead having electrodes disposed along its length to an electrical pulse generator for applying pulses of electrical potential to the electrodes; and
enclosing the connection between the lead and the pulse generator in a fluid impermeable housing so that the device is a single unit.

45. The method of claim 44, wherein the fluid impermeable housing is silicone.

46. A method of implanting an electrical nerve stimulation device in a human or animal body, the method comprising:
inserting a needle carrying a sheath into a subcutaneous region;
withdrawing the needle to leave the sheath in place in the subcutaneous region;
inserting an electrode lead of the electrical nerve stimulation device into the sheath; and
removing the sheath from the subcutaneous region to expose the electrode lead in the subcutaneous region.

47. The method of claim 46, wherein the sheath is torn from the electrode lead on removal of the sheath from the subcutaneous region.

48. A method of treating pain, the method comprising:
implanting an electrical nerve stimulation device in subcutaneous tissue of a human or animal body; and
applying electrical pulses to the tissue in which the device is implanted via an electrode lead of the device.

49. The method of claim 48, comprising implanting the electrical nerve stimulation device in subcutaneous tissue comprising fatty tissue found between the skin and the fascia and/or muscle tissue underlying the skin.

50. The method of claim 48, comprising implanting the electrical nerve stimulation device around 5 mm below the surface of the skin.

51. The method of claim 49, comprising implanting the electrical nerve stimulation device around 5 mm below the surface of the skin.

52. The method of claim 48, comprising implanting the electrode lead of the device in subcutaneous tissue under an area of skin at which the patient experiences allodynia or hyperalgesia.

53. The method of claim 48, comprising identifying an area of skin at which the patient experiences allodynia or hyperalgesia and implanting the electrode lead of the device in subcutaneous tissue under the identified area of skin.

54. The method of claim 48, comprising identifying an area of skin at which the patient experiences greatest allodynia or hyperalgesia and implanting the electrode lead of the device in subcutaneous tissue under the identified area of skin.

55. The method of claim 48, comprising implanting the lead in the inguinal canal.

56. The method of claim 48, wherein the electrode lead has electrodes disposed along its length and the electrical nerve stimulation device comprises:
an electrical pulse generator connected to the electrode lead for applying electrical pulses to the electrodes; and
a fluid impermeable housing enclosing the connection between the lead and the pulse generator so that the device is a single unit.

57. The method of claim 48, wherein the electrode lead has electrodes disposed along its length and the electrical nerve stimulation device comprises an electrical pulse generator connected to the electrode lead for applying electrical pulses to the electrodes, the distance along the lead between the pulse generator and the electrode closest to the pulse generator being less than around 5 cm.

58. The method of claim 48, wherein the electrode lead has electrodes disposed along its length and the electrical nerve stimulation device comprises:
an electrical pulse generator connected to the electrode lead for applying electrical pulses to the electrodes; and
a housing containing the pulse generator, which housing is substantially flat.

59. The method of claim 48, wherein the electrode lead has electrodes disposed along its length and electrical nerve stimulation device comprises:
an electrical pulse generator connected to the electrode lead for applying electrical pulses to the electrodes,
wherein the pulse generator is substantially flexible.

60. The method of claim 48, wherein the electrode lead has an electronically readable memory for storing data.

61. The method of claim 48, wherein the electrode lead has electrodes disposed along its length, the electrodes each comprising a group of electrical contacts.
